(19) United States
(12) Patent Application Publication
(10) Pub. No.: US 2008/0275488 A1
(43) Pub. Date: Nov. 6, 2008

(54) EXTENDED DURATION REMOVABLE MEDICAL FILTER

(76) Inventor: James A. Fleming, Bethlehem, PA (US)

Correspondence Address: RONALD L. HOFER, 122 LINDBERGH LANE, MOORESVILLE, NC, 28117, US

(21) Appl. No.: 11/742,702

(22) Filed: May 1, 2007

(51) Int. Cl. A61F 2/01

(52) U.S. Cl. 606/200

(57) ABSTRACT

A medical filter intended for placement inside a tubular body passage of a patient has a generally longitudinally extending central spine with a retrieval element at one end; and a plurality of filter sections attached to and spaced along the spine, each of the filter sections having a plurality of radial arms attached to the spine and extending radially outwardly with parallel end segments extending axially generally parallel to the longitudinal axis, each of the end segments ending in a tip which extends radially inwardly. Each filter section may also have a graft band attached to the parallel end segments of the radial arms.

### BACKGROUND AND SUMMARY OF THE INVENTION

The present invention relates to medical filters which are intended to be placed inside a blood vessel or other body passage for the purpose of intercepting thrombus or particles.

Medical filters, including vena cava filters, are emplaced inside blood vessels or other body passages for the purpose of intercepting thrombus or other particles while allowing free flow of blood in the vessels or other body fluid in the body passages. It has been found that certain features are desirable in such filters. For example, when used in a blood vessel, a filter must be effective to entrap thrombus, clots or other dangerous coagulations while allowing free flow of blood in the vessel. The filter should be emplaced with minimal trauma to the patient as by percutaneous delivery, and if it is desired to remove the filter, it is desirable that it can be removed with minimal trauma to the vessel. It is also desirable that the filter be adapted to properly adjust to the size of the vessel and that the filter remain effective during its time in place and that it be stable within the vessel.

Generally speaking, effective vena cava and other medical filters are known but there remains room for improved designs of such filters. For example, it would be desirable to have a filter designed to be implanted for an extended period of time and yet be removable with minimum trauma to the vessel. It would also be desirable to have a filter design which is adaptable to deployment in curved vessels. Also, while known filters are effective, there remains room for an improved filter design which enhances filtration. There also remains room for improved filter designs which are removable, if desired, yet resist migration within the vessel.

Accordingly, the present invention provides a medical filter which is suitable to be implanted in a vessel for an extended period of time yet can be removed with minimal trauma to the vessel. The medical filter of this invention is resistant to migration and has enhanced filtering characteristics. Furthermore, the filter is well suited for implantation in curved vessels.

Thus, in accordance with the present invention, a medical filter intended for placement inside a tubular body passage such as a vessel of a patient comprises:
a central spine extending along a longitudinal axis, said spine carrying a retrieval element at one end thereof; and
a plurality of filter sections attached to said spine and spaced apart longitudinally; each of said filter sections comprising a plurality of radial arms extending radially outwardly from said spine, each of said arms having an end segment bent to extend in an axial direction away from said retrieval element and generally parallel to said longitudinal axis, each said end segment having a tip bent radially inwardly.
   In a preferred embodiment of a medical filter, the generally parallel end segments of each arm of a filter section are removably attached to a graft material. Thus, each filter section has a cylindrical piece of graft material and radial arms that connect the graft material to the central spine. The design of the arms is such that they will detach easily from the vessel wall and the graft material for retrieval of the filter but resist migration in the vessel. The design will easily collapse into a cylindrical shape that can be loaded into a sheath and inserted percutaneously into a targeted vessel.

Further understanding of the present invention will be had from the following description taken in conjunction with the accompanying drawings and appended claims.

### BRIEF DESCRIPTION OF THE DRAWINGS

FIG. 1 is a perspective view of a preferred embodiment of a medical filter of the present invention shown in expanded form;

FIG. 2 is a side elevation of the preferred embodiment of FIG. 1;

FIG. 3 is a perspective view showing an alternative preferred embodiment of a medical filter of the present invention without graft material;

FIG. 4a and FIG. 4b are schematic views illustrating the steps in deploying a preferred embodiment of a medical filter of the present invention;

FIG. 5a, FIG. 5b and FIG. 5c are schematic views illustrating the steps of retrieving a preferred embodiment of a medical filter of the present invention;

FIG. 6 is a perspective view of an alternative preferred embodiment of a medical filter of the present invention;

FIG. 7 is a schematic view illustrating alternative designs of arms of preferred embodiments of medical filters of the present invention; and

FIG. 8 is a perspective view, broken away, showing a further alternative design of a preferred embodiment of a medical filter of the present invention.

### DETAILED DESCRIPTION OF THE PREFERRED EMBODIMENTS

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the preferred embodiment of the present invention are shown in use in a vein but may also be used in other vessels or body passages.

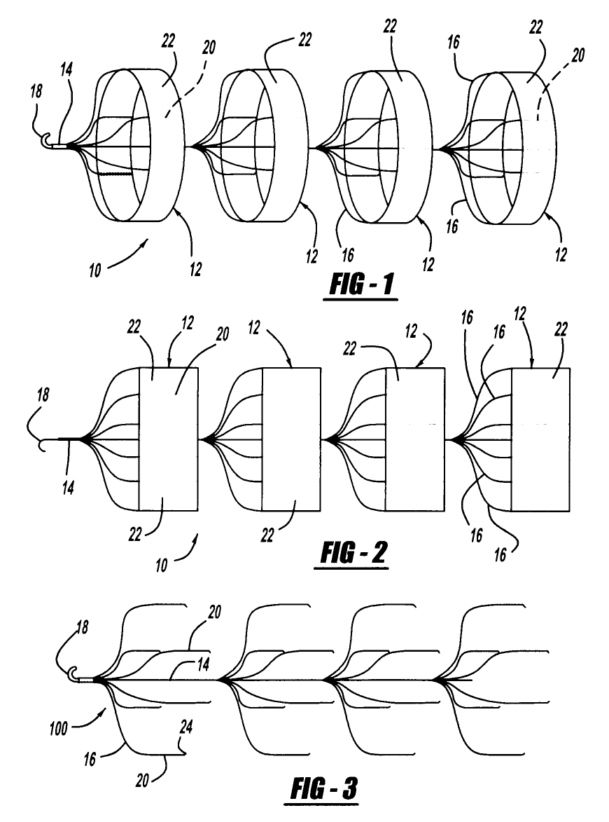

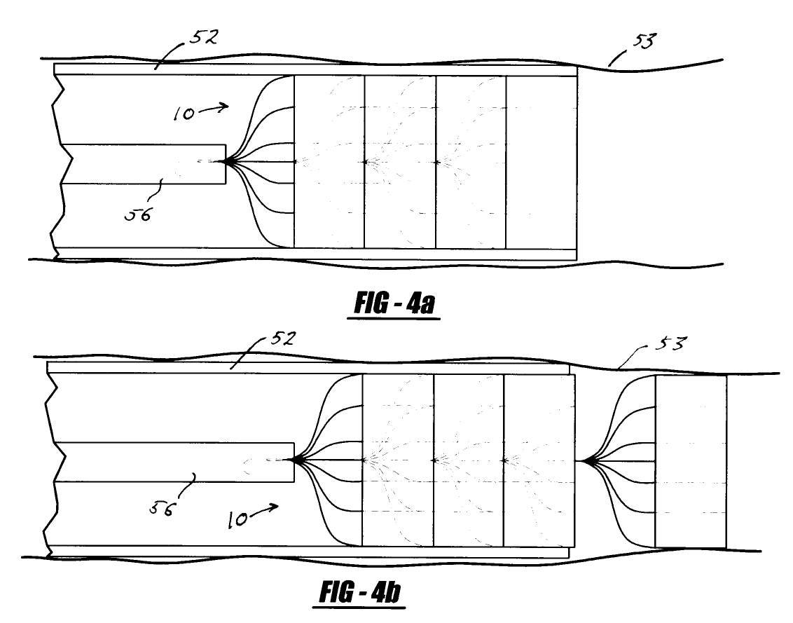

Now referring to FIGS. 1 and 2, a preferred embodiment of a medical filter of the present invention is shown and indicated generally by the numeral 10. Broadly speaking, filter 10 has a plurality of filter sections 12 which are attached to, and longitudinally spaced along, central spine 14 by a plurality of radial arms 16. Central spine 14 has a retrieval element 18 which can be a hook as shown in the figure or another element facilitating retrieval of medical filter 10. Retrieval element 18 may be a single hook as shown in the figures or may have a T-shape with twin hooks or any suitable shape which can operatively interact with a snare device. It will be appreciated by those skilled in the art that hooks and snares are well-known and that the particular retrieval means employed is subject to variation within the spirit of the present invention.

Each arm 16 is made of a resilient material, preferably a shape memory material, which tends to expand to the form illustrated in FIG. 1 but can be compressed radially to a smaller diameter to be carried in the lumen of a suitable delivery catheter as described in more detail below. Each arm 16 extends radially outwardly from spine 14 and has an end segment 20 which is bent from the radial portion of arm 16 to extend in an axial direction away from the end of spine 14 with retrieval element 18 and generally parallel to the longitudinal axis of filter 10 and spine 14. The preferred embodiment shown in FIGS. 1 and 2 has 6 arms 16 equally radially spaced about spine 14 but it will be appreciated by those skilled in the art that the exact number of arms 16 of each filter section 12 is not critical and that more or fewer arms 16 may be used for each filter section.

Each filter section 12 has a band 22 of graft material removably secured to parallel end segments 20 of arms 16. As shown in FIGS. 1 and 2, bands or bands 22 are tubular in form. Any suitable graft material may be used for band 22 so long as such material attaches well to the adjacent tissue wall, is biologically compatible for implantation, and is flexible so that medical filter 10 can be collapsed for delivery. Examples of suitable materials for band 22 include polyesters such as polyethyleneterephthalate, polytetraethylfluorine, polyethylene, polypropylene and other synthetic polymeric materials. The free end of each parallel end segment 20 terminates in a tip 24 which is bent radially inwardly so as to minimize the risk of puncturing a vessel wall.

Each parallel end segment 20 can be secured to graft bands 22 by stitching thereto or inserting segment 20 through the material of tube 22. Each arm 16 is also somewhat secured to each band 22 by each tip 24. However, it is important that there is little mechanical interference or other attachment of segment 20 to band 22 which would interfere with the withdrawal of segment 20 from band 22 in the axial direction leftward as viewed in FIGS. 1 and 2. It is desired that arms 16 will be easily detached from bands 22 so that they can be detached from the vessel wall without the graft material. On the other hand, each arm 16 resists movement in an associated vessel in the rightward direction as viewed in the figures because of the relatively large bend of each arm 16 extending radially outwardly from spine 14.

Filter sections 12 are longitudinally spaced along spine 14 to provide serial filtering and also to facilitate use of filter 10 in a curved vessel. Medical filter 10 is shown with four filter sections 12, but the exact number of filter sections may vary within the scope of the present invention. Each section 12 is preferably rotated slightly as shown in the figures to facilitate trapping of solid formations. As illustrated in FIGS. 1 and 2, each section 12 is rotated about 15° relative to the preceding section 12.

The spine and arms of filter 10 may be made of any suitable material using a variety of methods. Nitinol is a preferred material but elgiloy, cobalt chromium, stainless steel or suitable polymeric materials are examples of other materials that may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter. Thus, the material must also be sufficiently resilient to accomplish both compression in the delivery catheter and expansion upon ejection from the catheter.

Suitable methods of manufacture of the spine and arms of medical filter 10 include cutting a pattern into a tube to enable expansion of the tube into the desired body and struts. Another suitable method is forming the struts and body from separate strips or wires and then joining the respective parts together by suitable methods which are well known in the art.

Now referring to FIG. 3, an alternative preferred embodiment of a filter of the present invention is shown and indicated generally by the numeral 100. Medical filter 100 is analogous to; filter 10 except that filter 100 does not carry graft bands 22. In the case of filter 100, it is contemplated that parallel end segments 20 of each arm 16 will come into contacting relationship with the inner wall the vessel when implanted therein.

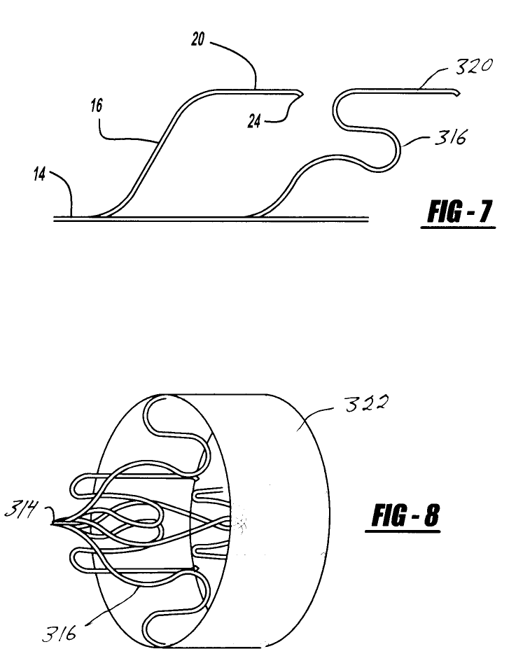

Having described the structure of medical filter 10, a preferred embodiment of the present invention, further understanding of the unique character and advantages of the present invention will be had by an understanding of its use. Now referring to FIGS. 4a and 4b, the steps of deploying filter 10 are illustrated. As indicated by numeral 50, filter 10 is shown compressed and loaded into sheath 52. It will be appreciated by those skilled in the art that filter 10 can be readily collapsed into a cylindrical shape that may be easily loaded into a sheath and inserted percutaneously into a targeted vessel such as vessel 53. Filter 10 is then deployed from sheath 52 and implanted in vessel 53 by pushing filter 10 out of the sheath with wire or tube 56 whereupon filter 10 expands into contact with the wall of vessel 54.

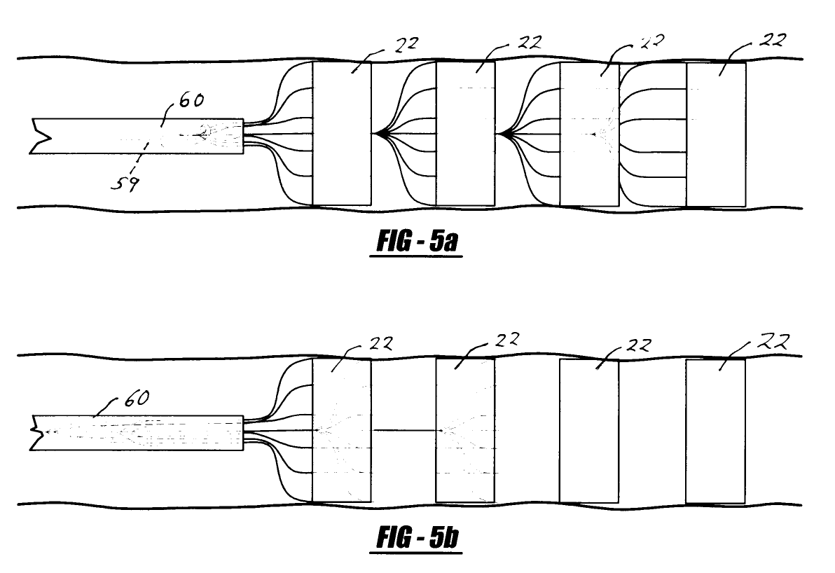

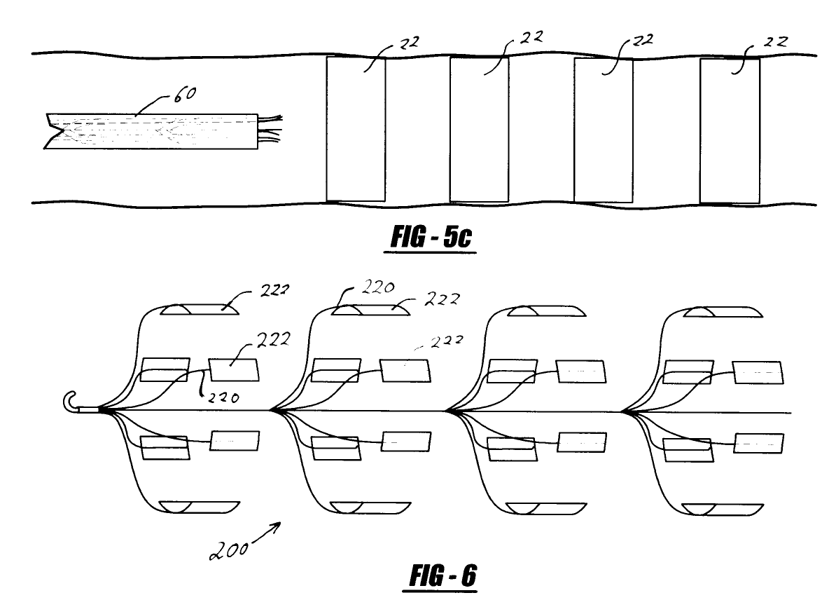

Retrieval of filter 10 from vessel 54 is illustrated in FIGS. 5a-5c. Thus filter 10 is shown as being pulled free from graph bands 22 by snare 59 of retrieval sheath 60. As filter 10 is pulled leftward as shown in FIG. 5, it is pulled free of graph bands 22 and collapses radially into sheath 60.

Now referring to FIG. 6, another alternative embodiment is shown and indicated generally by the numeral 200. Medical filter 200 is analogous to filter 10 except that instead of graft bands 22, medical filter 200 has graph pads 222. It will be appreciated that pads 222 function generally in the same manner as graph bands 22 and are intended to be incorporated into vessel tissue over a period of time just as in the case of bands 22. Similar to the removal of filter 10, when it comes time to remove filter 200, parallel end segments 220 of arms 216 can be easily withdrawn from pads 222 which have been grafted into adjacent tissue.

Now referring to FIGS. 7 and 8, further variations if arms 16 suitable for use in the present invention are illustrated. Thus, as illustrated diagrammatically in FIG. 7, arms 16 can have the shape shown in side elevation in FIG. 7 or can be shaped as illustrated therein by arms 316 to have a somewhat serpentine bend between spine 14 and parallel end segment 320. A filter section employing the arm shape of arm 316 is illustrated in FIG. 8 wherein spine 314 is connected to graft band 322 by arms 316. Of course, a single filter of the present invention may employ one or both of arms 16 and arms 316 or other similar constructions within the scope of the present invention.

While preferred embodiments of the present invention have been specifically described above, it will be appreciated by those skilled in the art that the present invention is subject to variations and modifications. For example, the filter may be cut from a single tube and have arms which are integral, i.e., one piece, with the spine. The filter may be tube-based or wire based or a combination. The curvature of the arms may vary. The number of arms and/or filter elements may vary. These and other modifications are contemplated to be within the scope of the present invention which is intended to be limited only by the following claims.

What is claimed is:

1. An elongated medical filter for placement inside a tubular body passage of a patient comprising:
(A) a central spine extending along the longitudinal axis of said filter, said spine carrying a retrieval element at one end thereof;
(B) a plurality of filter sections attached to said spine and spaced apart longitudinally thereon, each of said filter sections having a plurality of radial arms, each of said radial arms extending outwardly from said spine to a parallel end segment extending axially generally parallel to said longitudinal axis away from said retrieval element, each said end segment having a tip extending radially inwardly.

2. The medical filter of claim 1 wherein each said filter section has a graft band slidably removably attached to said parallel end segments.

3. The medical filter of claim 1 wherein said radial arms have a serpentine shape.

4. The medical filter of claim 1 wherein said filter has four filter sections.

5. The medical filter of claim 1 wherein each said filter section has a graft pad slidably attached to said parallel end segments.

6. The medical filter of claim 2 wherein said graft bands attached to said parallel end segments by threaded elements.

7. The medical filter of claim 2 wherein said graft bands are attached to said parallel end segments by said end segments extending through said graft bands.

8. The combination of a body vessel of a patient and implanted in said vessel, an elongated medical filter comprising:
(A) a central spine extending along the longitudinal axis of said filter, said spine carrying a retrieval element at one end thereof;
(B) a plurality of filter sections attached to said spine and spaced apart longitudinally thereon, each of said filter sections having a plurality of radial arms, each of said radial arms extending outwardly from said spine to a parallel end segment extending axially generally parallel to said longitudinal axis away from said retrieval element, each said end segment having a tip extending radially inwardly.

9. The combination of claim 8 wherein said vessel is curved.

10. The combination of claim 8 wherein said longitudinal axis of said filter is curved.

11. The combination of claim 10 wherein each said filter section has a graft band slidably removably attached to said parallel end segments.

12. The combination of claim 11 wherein said radial arms have a serpentine shape.

13. The combination of claim 11 wherein said filter has four filter sections.

14. The combination of claim 11 wherein each said filter section has a graft pad slidably attached to said parallel end segments.

15. The combination of claim 11 wherein said graft bands attached to said parallel end segments by threaded elements.

16. The combination of claim 11 wherein said graft bands are attached to said parallel end segments by said end segments extending through said graft bands.
